(19) United States
(12) Patent Application Publication
(10) Pub. No.: US 2007/0167950 A1
(43) Pub. Date: Jul. 19, 2007

(54) SYSTEM AND METHOD FOR ATTACHING SOFT TISSUE TO BONE

(76) Inventors: Joseph C. Tauro, Brick, NJ (US); Michael Green, Pleasanton, CA (US); Bart Bojanowski, San Jose, CA (US); Tate Scott, San Diego, CA (US); Malcolm Heaven, Dana Point, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/614,907

(22) Filed: Dec. 21, 2006

Related U.S. Application Data
(60) Provisional application No. 60/753,445, filed on Dec. 22, 2005.

(51) Int. Cl. A61B 17/58

(52) U.S. Cl. 606/73

(57) ABSTRACT

Disclosed herein are methods and devices for securing soft tissue to a rigid material such as bone. A bone anchor is described that comprises a base and a top such that suture material may be compressed between surfaces on the base and top to secure the suture to the anchor. Also described is an inserter that can be used to insert the bone anchor into bone and move the anchor top relative to the anchor base to clamp suture material there between. Also described is a soft-tissue and bone piercing anchor and associated inserter. Methods are described that allow use of the bone anchors to provide multiple lengths of suture material to compress a large area of soft tissue against bone.

### RELATED APPLICATION

This application claims the benefit of U.S. Provisional Application No., 60/753,445, filed Dec. 22, 2005, which is incorporated herein by reference in its entirety.

### BACKGROUND OF THE INVENTION

1. Field of the Invention

The present invention relates to medical devices and procedures. More particularly, the present invention relates to devices and methods for securing soft tissue to a rigid material such as bone.

2. Description of the Related Art

There are several medical procedures where a surgeon needs to attach soft tissue such as tendons or other soft connective tissue to bone. One common example is a torn rotator cuff, where the supraspinatus tendon has separated from the humerus causing pain and loss of ability to elevate and externally rotate the arm. To repair a torn rotator cuff, typically a surgical procedure is used to suture the torn tendon to the bone using a variety of methods. Some procedures utilize large incisions and involve complete detachment of the deltoid muscle from the acromion. Small diameter holes are made in the bone for passing suture material through the bone to secure the tendon. Such large incision procedures are traumatic, causing prolonged pain and recovery time. Other procedures make small incisions and use arthroscopic techniques to attach sutures using either small diameter holes or a bone anchor. However, it is difficult to manipulate sutures within the surgical site using arthroscopic techniques. In addition, when knot tying is used to secure the suture to a bone anchor, it is difficult to properly adjust the tension of the suture while tightening the knot. Similarly, when the suture is attached to a bone anchor prior to insertion of the anchor into the bone, it is difficult to judge the appropriate point of attachment so that the suture will be properly tensioned upon insertion of the bone anchor into the bone. Thus, there is a need for methods and devices that allow easy arthroscopic attachment of a suture to a bone anchor after the anchor is inserted into the bone without the use of knot tying.

### SUMMARY OF THE INVENTION

The present invention is particularly suited for use in arthroscopic procedures, including but not limited to rotator cuff surgery. More broadly, it can be used in any procedure in which it is desired to fix a suture to a solid object without tying of knots, including not only arthroscopic procedures, but also open surgery, and can be used for such diverse purposes as bladder neck suspension, tendon and ligament affixation or repair, prosthetic attachment, and rotator cuff repair.

In one embodiment, the invention includes an anchor for securing a suture to bone, including an anchor base adapted to be securely fixed into the bone and a suture securing mechanism coupled to the anchor base and positioned proximally relative to the anchor base, the mechanism adapted to receive and secure a suture moved laterally into the

In another embodiment, the invention includes an anchor for securing a suture to bone, including an anchor base adapted to be securely fixed into the bone, a first surface coupled to the anchor base and positioned proximally relative to the anchor base, and a second surface coupled to the anchor base and positioned proximally relative to the anchor base, wherein the first and second surfaces are adapted to be relatively positioned in at least two configurations, one of the configurations such that a gap is present between the first and second surfaces so that the suture can be positioned between the first and second surfaces by moving the suture laterally into the gap, and the other of the configurations such that the first and second surfaces are in close proximity so that the suture can be securely clamped between the first and second surfaces.

In another embodiment, the invention includes a method of attaching soft tissue to bone, including passing a length of suture over the soft tissue, inserting an anchor into the bone, and securing the length of suture to the anchor after the inserting without passing an end of the length of suture through any aperture in the anchor and without tying any knots.

In another embodiment, the invention includes a method of attaching soft tissue to bone, including inserting a first anchor through the soft tissue, wherein the first anchor comprises a length of suture fixedly secured to the first anchor prior to insertion, inserting the first anchor into the bone, passing the length of suture over the soft tissue, and fixedly securing, after the passing, the length of suture to a second anchor.

In another embodiment, the invention includes a method of attaching soft tissue to bone, the soft tissue comprising a first surface adjacent to the bone's surface and a second surface opposite the first surface, the method including inserting a first portion of a length of suture into the second surface of the soft tissue, passing a second portion of the length of suture over the second surface of the soft tissue, inserting a first anchor with no suture coupled thereto into the bone, and fixedly securing the length of suture to the inserted first anchor, with the proviso that no part of the first portion of the length of suture is passed out of the second surface of the soft tissue.

In another embodiment, the invention includes a method of attaching soft tissue to bone, including inserting a first anchor with a length of suture pre-coupled thereto through the soft tissue, inserting the first anchor into the bone, inserting a second anchor with no suture coupled thereto into bone, passing the length of suture over the soft tissue, and fixedly securing the length of suture to the inserted second anchor.

In another embodiment, the invention includes a method of attaching soft tissue to bone, the method including inserting a first, second, and third anchor into the bone, fixedly securing a first length of suture over the soft tissue to the first and second anchors, and fixedly securing a second length of suture over the soft tissue to the first and third anchors.

In another embodiment, the invention includes an anchor for securing a suture to bone, the anchor including an anchor base adapted to be securely fixed into the bone, the anchor base comprising a first proximal surface and an anchor top, the anchor top comprising a distal member coupled to the anchor base and a first proximal member comprising a first distal surface, wherein the anchor top is adapted to couple to the anchor base in at least two configurations, one of the configurations such that the first distal surface is above the bone's surface when the anchor base is securely fixed into the bone, such that a suture can be freely passed between the first proximal and first distal surfaces above the bone's surface, and the other of the configurations such that the first distal surface is in close proximity to the first proximal surface, such that a suture can be securely clamped between the first proximal and first distal surfaces.

In another embodiment, the invention includes an anchor for securing a suture to bone, the anchor including a substantially hollow cylinder comprising an open end and comprising a portion of its walls cut in such a manner so as to allow the cylinder to deform under stress and form lateral protrusions, a substantially pointed tip coupled to the cylinder opposite the open end, wherein the pointed tip is adapted to pierce the bone, and a suture receiver coupled to the pointed tip and positioned within the substantially hollow cylinder so that a suture may be attached to the suture receiver and extend through the cylinder and out of the open end.

In another embodiment, the invention includes a suture anchor that comprises a distal portion configured to engage bone and a proximal portion comprising at least two separate suture capture mechanisms, each configured to secure a separate suture without tying any knots. In one embodiment, each suture capture mechanism comprises first and second surfaces configured to clamp suture therebetween. In one embodiment, the two suture capture mechanisms are configured to secure sutures at different times. In one embodiment, the proximal portion comprises a first, second, third, and fourth surface, wherein the proximal portion is configured to secure at least one suture by clamping between the first and second surfaces and is configured to secure at least one other suture by clamping between the third and fourth surfaces. In one embodiment, each suture capture mechanism defines a suture capture zone configured such that a suture may be moved laterally into the suture capture zone.

In another embodiment, the invention includes a suture anchor that comprises a distal portion configured to engage bone, the distal portion comprising a proximally facing surface, a proximal portion comprising a distally facing surface, and a medial portion comprising a proximally facing surface and a distally facing surface, wherein the anchor is configured to secure at least one suture by clamping between the proximally facing surface of the distal portion and the distally facing surface of the medial portion and adapted to secure at least one other suture by clamping between the distally facing surface of the proximal portion and the proximally facing surface of the medial portion. In one embodiment, the anchor is configured such that a suture can be clamped between the distally facing surface of the medial portion and the proximally facing surface of the distal portion prior to a suture being clamped between the proximally facing surface of the medial portion and the distally facing surface of the proximal portion. In one embodiment, the proximal portion comprises a shaft positioned within a bore in the distal portion. In one embodiment, the shaft is movable within the bore. In one embodiment, the shaft extends through a bore in the medial portion. In one embodiment, the medial portion is movable relative to the shaft. In one embodiment, the medial portion comprises a shaft positioned within a bore in the distal portion. In one embodiment, the proximal portion comprises a shaft positioned within a bore in the medial portion.

In another embodiment, the invention includes a method of attaching two or more sutures to a suture anchor, including moving a first suture laterally into a first suture capture mechanism on the suture anchor, moving a second suture laterally into a second suture capture mechanism on the suture anchor, actuating the first suture capture mechanism to secure the first suture to the suture anchor, and actuating the second suture capture mechanism to secure the second suture to the suture anchor. In one embodiment, actuating the first suture capture mechanism comprises clamping the first suture between first and second surfaces of the first suture capture mechanism. In one embodiment, actuating the second suture capture mechanism comprises clamping the second suture between first and second surfaces of the second suture capture mechanism. In one embodiment, the first and second suture capture mechanisms are actuated simultaneously. In one embodiment, the first suture capture mechanism is actuated before actuating the second suture capture mechanism. The second suture is moved laterally into the second suture capture mechanism after the first suture capture mechanism is actuated.

In another embodiment, the invention includes a method of attaching soft tissue to bone, including inserting a first, second, and third anchor into the bone, securing a first suture between the first and second anchors over the soft tissue, wherein securing the first suture to the first anchor comprises laterally moving the first suture into a first suture capture mechanism, and securing a second suture between the first and third anchors over the soft tissue, wherein securing the second suture to the first anchor comprises laterally moving the second suture into a second suture capture mechanism. In one embodiment, the first suture is secured to the first anchor prior to securing the second suture to the first anchor. In one embodiment, the first and second sutures are simultaneously secured to the first anchor. In one embodiment, securing the first suture to the first anchor comprises clamping the first suture between a first and second surface of the first suture capture mechanism. In one embodiment, securing the second suture to the first anchor comprises clamping the second suture between a first and second surface of the second suture capture mechanism.

In another embodiment, the invention includes a suture anchor, comprising an anchor base configured to engage bone, wherein the anchor base comprises a bore having an opening on a proximal end of the anchor base, wherein the inner surface of the bore comprises angled protrusions extending in a direction away from the opening and an anchor top comprising a shaft configured to fit within the bore, wherein the shaft comprises circumferential grooves in its outer surface, wherein the circumferential grooves have a profile shaped such that when the angled protrusions extend into the grooves, the protrusions are not significantly deflected from their orientation prior to insertion of the shaft into the bore. In one embodiment, the circumferential grooves comprise surfaces angled relative to the outer surface of the shaft at approximately the same angle as the protrusions such that when the protrusions extend into the grooves, the protrusions are aligned with angled surfaces. In one embodiment, the angle of the angled surfaces is approximately 45°. In one embodiment, the anchor top comprises a member positioned on the proximal end of the shaft having a distally facing surface such that a suture may be clamped between the distally facing surface and the anchor base when the shaft is inserted into the bore.

In another embodiment, the invention includes a suture anchor that comprises an anchor base configured to engage bone, wherein a proximal end of the anchor base comprises a circular groove and an anchor top comprising a circular lip configured to fit inside the circular groove such that a suture can be clamped between the surfaces of the lip and the surfaces of the groove, wherein a least one surface of the lip or the groove comprise teeth configured to engage the suture.

In another embodiment, the invention includes a suture anchor inserter that comprises a handle, a tube coupled to the handle configured to engage an actuatable suture anchor, a shape memory alloy wire coupled to the handle and coupled to the actuatable suture anchor, and a heating element configured to heat the shape memory alloy wire, thereby causing the wire to contract relative to the tube and actuate the suture anchor. In one embodiment, the shape memory alloy wire is a nitinol wire. In one embodiment, the actuatable suture anchor comprises an anchor base and an anchor top configured to actuate by pressing the anchor top down onto the anchor base. In one embodiment, the actuatable suture anchor comprises a deformable anchor configured to deform under axial force to form lateral wings.

In another embodiment, the invention includes a suture anchor inserter that comprises a handle, a tube coupled to the handle configured to engage an actuatable suture anchor, a wire coupled to the handle and coupled to the actuatable suture anchor, and a motor configured to move the wire axially relative to the tube, thereby actuating the suture anchor. In one embodiment, the actuatable suture anchor comprises an anchor base and an anchor top configured to actuate by pressing the anchor top down onto the anchor base. In one embodiment, the actuatable suture anchor comprises a deformable anchor configured to deform under axial force to form lateral wings.

### BRIEF DESCRIPTION OF THE DRAWINGS

FIG. 1 is a perspective view depicting attaching soft tissue to bone using a single bone anchor and a stitch.

FIG. 2 is a perspective view depicting attaching soft tissue to bone using a two bone anchors with a suture stretched there between.

FIGS. 3A-3C is a perspective view depicting various geometries of bone anchors and suture patterns for attaching soft tissue to bone.

FIGS. 4A-4D depicts the base of a two-part suture anchor that can be inserted into bone.

FIGS. 5A-5C depicts the top of a two-part suture anchor.

FIG. 6 is a perspective view of the distal portion of one embodiment of an anchor top.

FIG. 7 is a cross-sectional view of part of the proximal portion of one embodiment of an anchor top.

FIGS. 8A and 8B are cross-sectional views depicting the suture anchor top of FIGS. 5A-5C inserted into the suture anchor bottom of FIGS. 4A-4D.

FIGS. 9A and 9B are cross-sectional views depicting multiple-part suture anchors providing multiple suture attachment zones.

FIGS. 10A and 10B are perspective views depicting a suture anchor inserter.

FIG. 11 is a perspective view depicting components on a suture anchor inserter for attaching to bone and manipulating a suture anchor.

FIGS. 12A-12E are perspective views depicting manipulation of a suture anchor using a suture anchor inserter to insert the suture anchor into bone and attach suture material to the suture anchor.

FIG. 13 is a perspective view depicting manipulation of a suture anchor using a suture anchor inserter including a sleeve to guide suture placement.

FIGS. 14A and 14B are perspective views depicting a piercing bone anchor in an un-deployed (FIG. 14A) and deployed (FIG. 14B) state.

FIG. 15 is a perspective view depicting a piercing bone anchor employing a shoulder for manipulating soft tissue.

FIGS. 16A-16F are perspective views depicting a method for attaching soft-tissue to bone using a piercing bone anchor and a suture capturing anchor.

FIG. 17 are perspective views depicting various markings on a bone anchor inserter for indicating depth of anchor insertion.

### DETAILED DESCRIPTION OF THE CERTAIN EMBODIMENTS

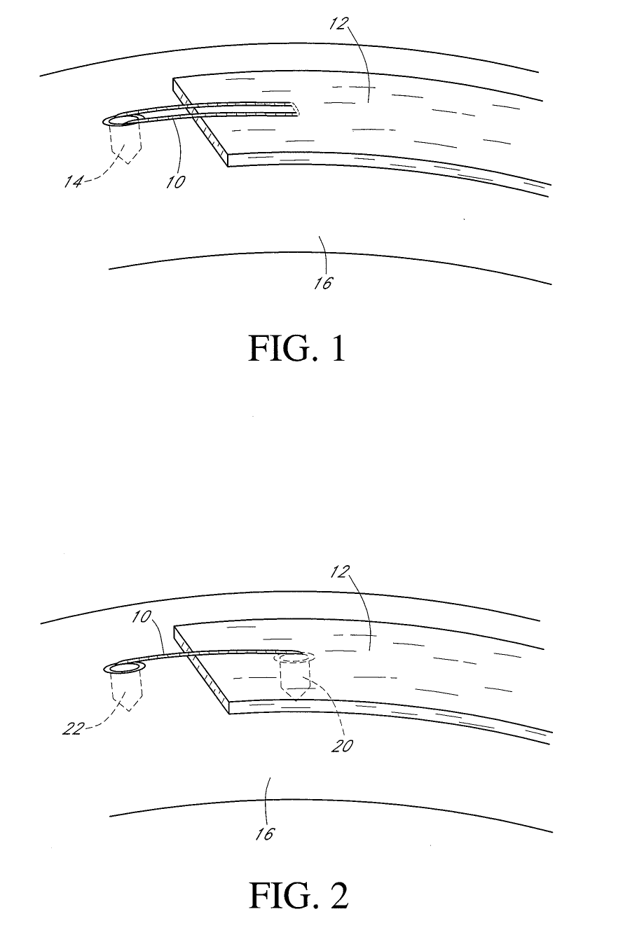

In various embodiments, soft tissue may be attached to bone utilizing one or more bone anchors with suture attached thereto. As used herein, “suture” refers to any flexible structure that can be stretched between two or more anchors and includes, without limitation, traditional suture material, single or multiple stranded threads, or a mesh structure. In some embodiments, suture is passed over the top of the soft tissue so that the suture can press the soft tissue against the bone. In one embodiment, a length of suture is attached to a single bone anchor. One non-limiting example, depicted in FIG. 1, includes stitching the suture 10 to the soft tissue 12, such as by an incline mattress stitch, and then securing the suture 10 to the single bone anchor 14 that is inserted into the bone 16. However, in other embodiments, a length of suture is attached to multiple bone anchors. The use of multiple bone anchors increases the footprint over which the suture material presses the soft tissue against bone. One non-limiting example, depicted in FIG. 2, includes two bone anchors. One anchor 20 is positioned in a medial location underneath the soft tissue 12 and a second anchor 22 is positioned lateral to the soft tissue 12. The suture 10 is attached to both anchors.

In one embodiment, the suture 10 is attached to the lateral bone anchor 22 only after the medial bone anchor 20 is inserted and the suture 10 is passed over the soft tissue 12. In one embodiment, the suture 10 is attached to the medial bone anchor 20 prior to insertion of the medial bone anchor 20. Thus, in this embodiment, the surgeon does not need to pass the suture through the soft tissue 12 from beneath the soft tissue 12. In one embodiment, the procedure involves inserting the medial bone anchor 20 with suture 10 pre-attached through the soft tissue 12. The medial bone anchor 20 may then be moved laterally relative to the bone 16 in order to pull the soft tissue 12 laterally relative to the bone 16. After appropriate positioning of the soft tissue 12, the medial bone anchor 20 may then be inserted into the bone 16. The lateral bone anchor 22 may then be inserted into the bone 16. The suture 12 may then be passed over the soft tissue 12 and attached to the lateral bone anchor 22. In some embodiments, a lateral bone anchor 22 is provided to which suture 12 can be attached without tying any knots or without passing the suture 12 through any aperture in the lateral bone anchor 22.

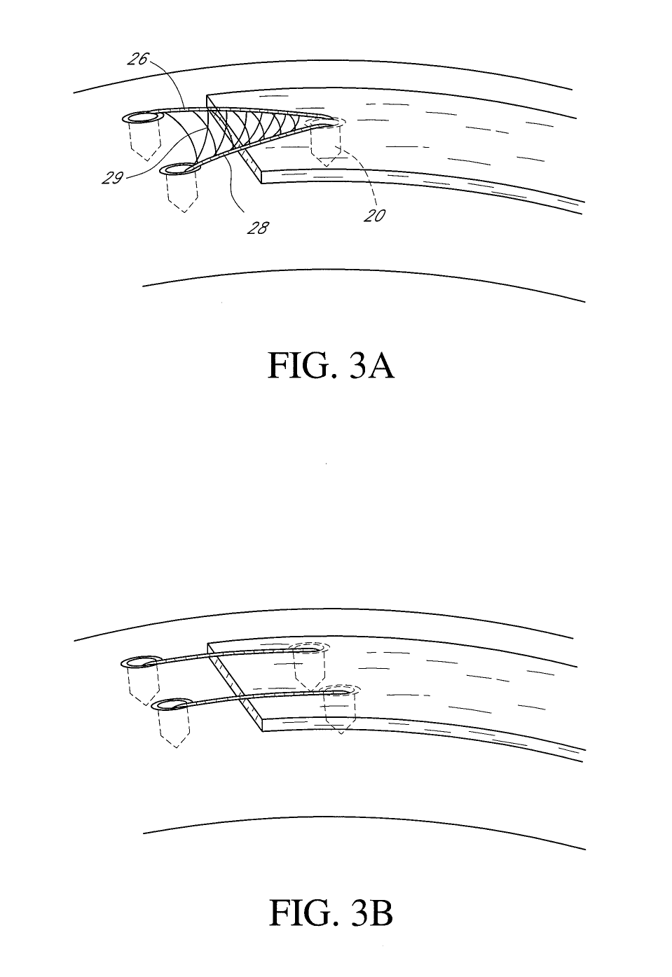

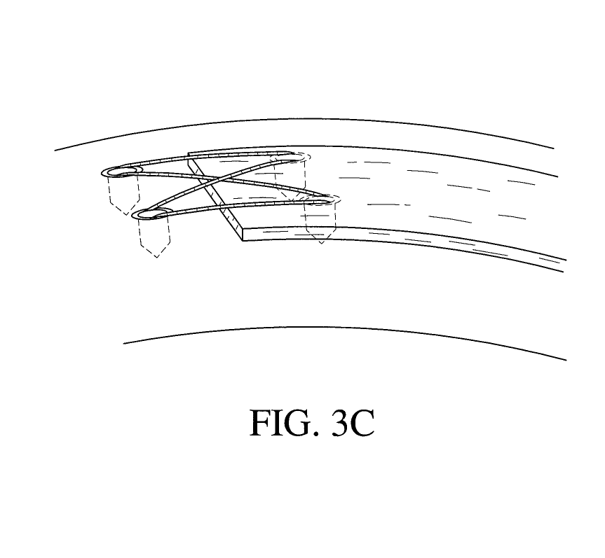

In some embodiments, multiple anchors and multiple suture lengths may be used to provide a wider area of pressure of the soft tissue against bone. For example, as depicted in FIG. 3A, three anchors are used with two lengths of suture 26 and 28. Alternatively, a mesh structure 29 may be stretched between the three anchors. In another example, as depicted in FIG. 3B, four anchors are used with two lengths of suture. In still another example, as depicted in FIG. 3C, four anchors are used with four lengths of suture. In some embodiments, the individual suture lengths may be part of a larger continuous suture. For example, in FIG. 3A, the suture lengths 26 and 28 may be part of a larger length of suture such that the lengths 26 and 28 are joined at medial bone anchor 20. Those of skill in the art will appreciate that there are any number of anchor and suture geometries that can be used.

In some embodiments, the medial bone anchors 20 are designed so that they can be easily pierced through the soft tissue 12 and bone 16. In some embodiments, the lateral bone anchors 22 are designed so that they can easily capture suture material after insertion of the bone anchors 22. Together, these design features provide a suturing system and method that provides an increased footprint of suture pressure against the soft tissue 12 and ease of implementation for a surgeon. For example, in some embodiments, the entire procedure may be done arthroscopically, with the surgeon needing only to insert the medial bone anchor 20 with suture optionally pre-attached through a first port, insert the lateral anchor 22 through a second port, pass the suture over the soft tissue 12 by capturing it from within the second port, and securing the suture to the lateral anchor 22. Accordingly, described below are certain embodiments of anchors adapted to capture suture material and anchors adapted to easily pierce through soft tissue and bone.

Suture Capturing Anchor

One embodiment is a bone anchor that allows easy capturing and securing of a suture after the bone anchor is inserted into the bone. In one embodiment, the bone anchor includes a suture securing mechanism positioned on the proximal end of the bone anchor (i.e., the end nearest the surface of the bone and the surgeon). In one embodiment, the suture securing mechanism allows a suture to be moved laterally into the mechanism. By “laterally,” it is meant that the suture can be moved into the mechanism by moving the suture in a direction that is generally perpendicular to the axis of the suture. In other words, the suture can be moved into the mechanism without threading an end of the suture into the mechanism. In one embodiment, the suture can be fixedly secured within the mechanism without tying any knots. By “fixedly secured,” it is meant that the suture within the securing mechanism cannot be easily moved relative to the bone anchor.

One embodiment is a bone anchor that allows easy attachment of suture material by clamping the suture material between two surfaces on the bone anchor. The bone anchor may be configured such that the bone anchor is inserted into the bone without the suture material attached. The two surfaces of the suture securing mechanism may be spaced apart so as to form a gap between the surfaces. The suture material may be passed between the two surfaces and tensioned as desired followed by clamping of the two surfaces together, thereby clamping the suture material therebetween.

In one embodiment, the bone anchor consists of two parts: an anchor base and an anchor top. The anchor base may be designed to be inserted into a hole in the bone with a proximal surface facing up. The anchor top may be coupled to the anchor base via a distal member. A proximal member on the anchor top may have a distal surface facing down toward the proximal surface on the anchor base. The coupling of the anchor top to the anchor base may be such that the anchor top can move relative to the anchor base such that it can be positioned in one configuration where there is space between the proximal surface on the anchor base and the distal surface on the proximal member of the anchor top. In another configuration, the proximal member of the anchor top may be position such that there is very little space, if any, between the proximal surface on the anchor base and the distal surface on the proximal member of the anchor top. Thus, in the first configuration, suture material may be easily passed between the two surfaces and tensioned as desired. In the second configuration, the suture material may be clamped between the two surfaces such that the suture is secured to the bone anchor.

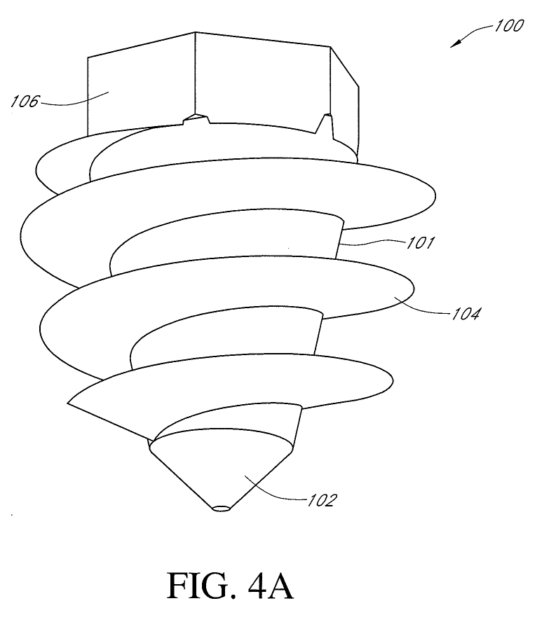

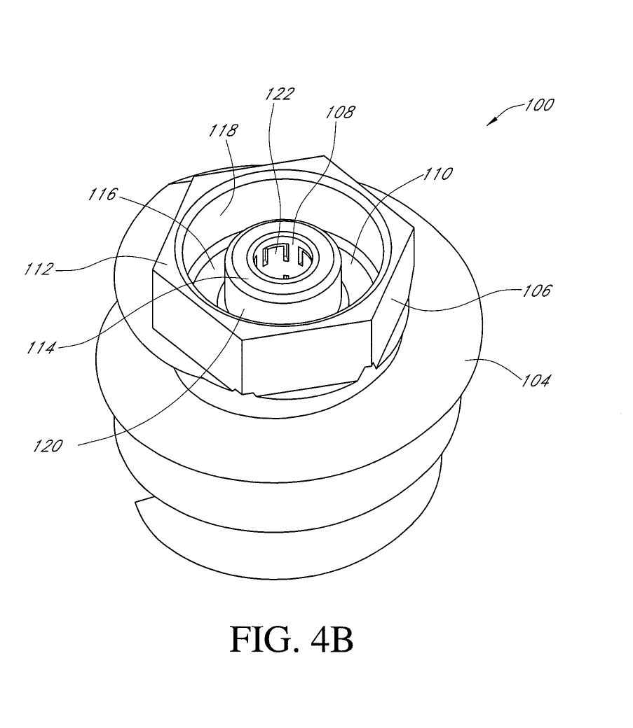

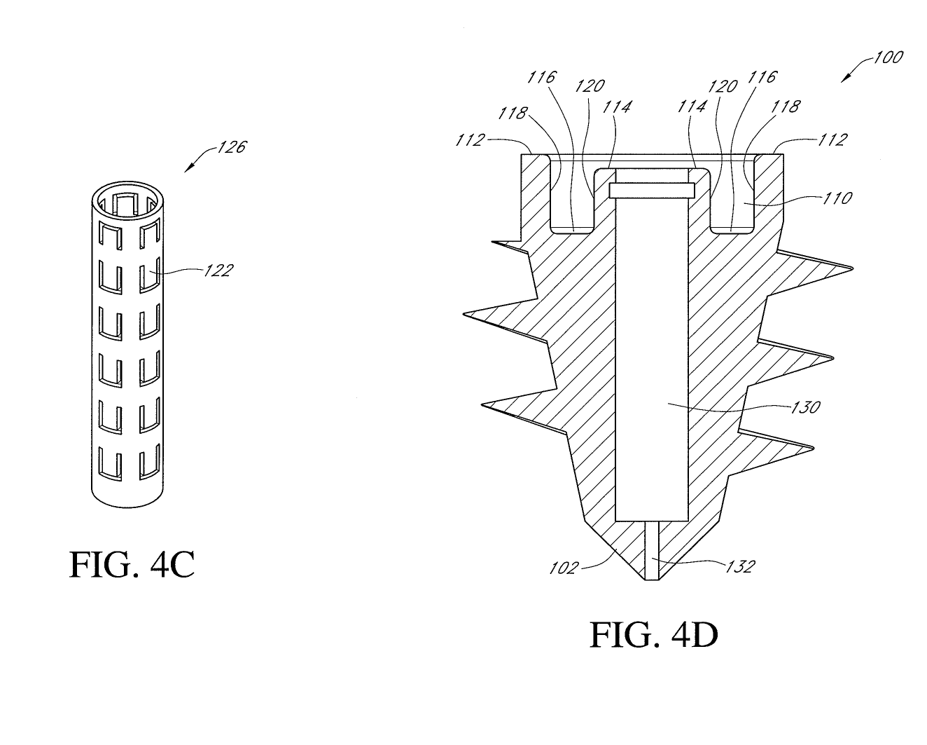

One embodiment of an anchor base 100 is depicted in FIGS. 4A through 4D. FIG. 4A is a perspective view showing the side 101 and bottom 102 of the anchor base 100. The bottom 102 of the anchor base 100 may advantageously be tapered to facilitate insertion of the anchor base 100 into bone. In some embodiments, a hole is predrilled into the bone to facilitate insertion of the anchor base 100. In other embodiments, the anchor base 100 is forced directly into the bone, thereby creating the hole. The sides 101 of the anchor base 100 comprise threads 104 so that the anchor base 100 may be inserted into bone using a screwing action. In some embodiments, the anchor base 100 may be tapped to start the threads 104 into the bone followed by screwing the anchor base 100 into the bone. When the hole in the bone is pre-drilled, the hole is advantageously drilled with a diameter smaller than the diameter of threads 104 so that the threads engage the bone through the sides of the hole. It will be appreciated that means other than threads may be used to secure the anchor base 100 to bone. For example, angled protrusions may be used that provide greater resistance to removal of the anchor base 100 than to insertion. The protrusions may be static or deployable once the anchor is inserted.

The top of anchor base 100 preferably includes a structure 106 for facilitating the driving or screwing of the base 100 into the bone. In the illustrated embodiment, this comprises a hex nut structure 106 that facilitates engagement with a hex nut driver for screwing the anchor base 100 into the bone. It will be appreciated that other structures known in the art for engaging tools used for screwing action may be used instead of hex nut structure 106, and that this structure can be indented into or extending out from the top of the anchor base 100, or can alternatively be formed on the sides of the anchor base 100.

With reference to FIG. 4B, which is a perspective view of the top and side of anchor base 100, the top (proximal end) comprises a hole 108 in the center for receiving the anchor top, which is described below. The top of anchor base 100 also contains a suture gripping structure such as a circular groove 110 that may be concentric with hole 108. Because of groove 110, the proximal surface of anchor base 100 is not flat and comprises top surfaces 112 and 114, bottom surface 116, and side surfaces 118 and 120. In some embodiments, some or all of these surfaces may be textured or otherwise altered so as to inhibit movement of suture material pressed against the surfaces. Suitable structures for inhibiting suture movement include, but are not limited to, teeth on the surfaces, bead blasting of the surfaces, etching the surfaces such as through a chemical etch process, use of a rubber o-ring such as within the groove 100, and various coatings such as a hydroxyapatite coating, grit coatings, or microencapsulated biocompatible adhesives. Although a grooved surface is illustrated, it will be appreciated that other shapes for the proximal surface of anchor base 100 are also contemplated, including multiple concentric grooves, a series of protruding ridges, a “vee” shaped channel, or any other suitable structure that permits a suture to be securely locked against the top or proximal end of the anchor base 100.

Hole 108 in anchor base 100 is an opening into a central (“axial”) bore into the anchor base 100. The sides of the central bore preferably include structures for gripping something inserted into the central bore, such as ratchet structures 122. FIG. 4C show a central ratchet bushing 126 that fits within the central bore and contains the ratchet structures 122. In the embodiment of FIG. 4C, the ratchet structures 122 are constructed by cutting U shaped cuts into bushing 126. The U shaped cuts then define tabs that make up the ratchet structures 122. It will be appreciated that other shapes and methods for making ratchet structures may be used. The purpose of ratchet bushing 126 is to receive the anchor top and secure it to the anchor base 100. It will be appreciated that other methods of securing the anchor top to the anchor base 100 may be used, such as a frictional fit or threading. Furthermore, the anchor top may be coupled to the anchor base 100 using means other than hole 108 and bushing 126. For example, the anchor top may be coupled via structures at the perimeter rather than the center or by a hinge.

FIG. 4D depicts a cross section through the center of anchor base 100. This view illustrates central bore 130 and groove 110. The proximal surfaces 112, 114, 116, 118, and 120 are also apparent. Central bore 130 preferably does not extend all the way through the anchor base 100. Instead, a smaller bore 132 is present at the distal end 102 of the anchor base 100. Smaller bore 132 is used to receive a wire connected to an anchor inserter. It will be appreciated that other structures than bore 132 may be used for attaching the wire and that other means than a wire may be used to secure the anchor to the anchor inserter.

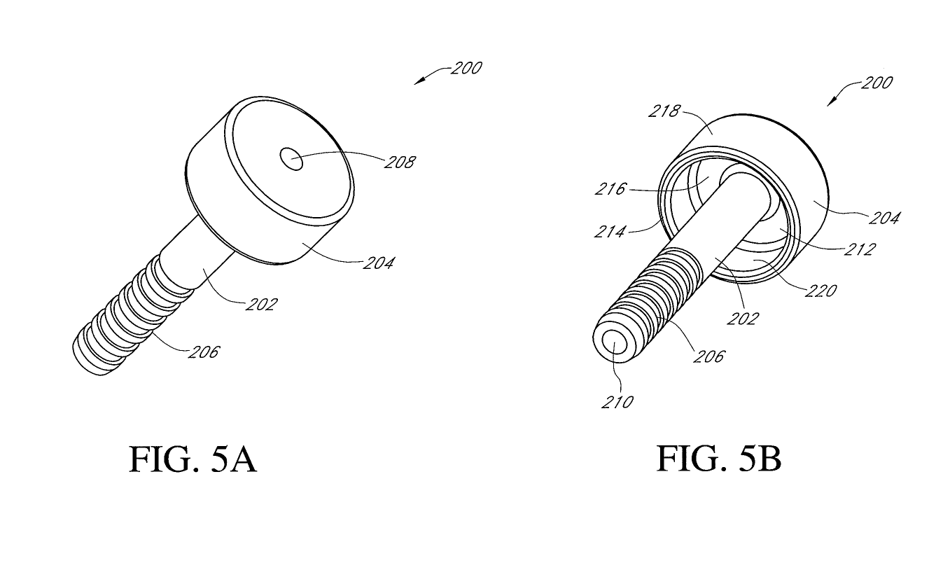

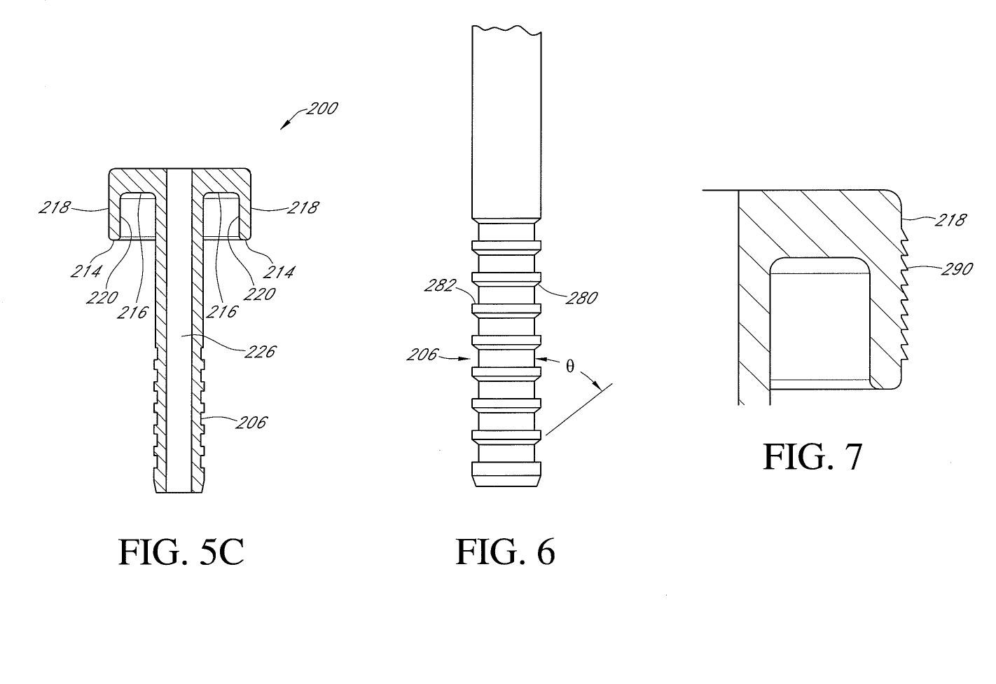

FIGS. 5A through 5C illustrate one embodiment of an anchor top 200. FIG. 5A provides a perspective view of the side and top of the anchor top 200 and FIG. 5B provides a perspective view of the side and bottom of the anchor top 200. Anchor top 200 has two members, a distal member 202 and a proximal member 204. The distal member 202 comprises an elongated shaft, the longitudinal direction of which shall be considered to run along the axis of the distal member 202. A series of grooves or other mating or locking surfaces or structures 206 exist along a portion of the outside surface of the shaft. The distal member 202 is designed to be inserted into the central bore 130 of the anchor base 100. The ratchet structures 122 in the anchor base 100 engage grooves 206 to couple the anchor top 200 to the anchor base 100. The ratchet structures 122 are oriented such that the distal member 202 can be easily moved in the distal direction in central bore 130 with the ratchet structures 122 snapping into the grooves 206 as the distal member 202 is moved downward. However, when the ratchet structures 122 are snapped into grooves 206, proximal movement of distal member 202 is inhibited. Thus, the anchor top 200 may be ratcheted down into anchor base 100. Because the ratchet structures 122 exist along substantially the entire surface of the central bore 130 (see FIG. 4C), the anchor top 200 may be coupled to the anchor base 100 in several positions. In other words, in one embodiment the anchor top 200 need not be ratcheted into the anchor base 100 as far as it will go for it to be secured to the anchor base 100. In alternative embodiments, structures may be used that allow the anchor top 200 to be reversibly moved into the anchor base 100 such that if suture tension needs to be corrected, it can be unclamped and then reclamped. For example, instead of grooves 206 in the anchor top 200 and ratchet structures 122 in the anchor base, threading may be used so that the anchor top 200 may be screwed into the anchor base 100 as well as unscrewed.

In one embodiment, the grooves 206 in the distal member 202 are shaped in such a way so as to increase the force required to pull the distal member 202 from the central bore 130 of the anchor base 100. For example, it has been discovered that one problem with rectangular shaped grooves 206, such as depicted in FIG. 5C, is that the ratchet structures 122 (see FIG. 4C) contact the distal facing corners of the grooves 206. This contact causes the ratchet structures 122 to partially depress such that they do not fully engage the grooves 206. Accordingly, in one embodiment, the distal facing corners of the grooves 206 are flattened to limit their interference with the ratchet structures 122 fully engaging the grooves. For example, the distal facing sides in the grooves 206 may be rounded or angled. One such embodiment is depicted in FIG. 6. In this example, the distal facing sides 280 of the grooves 206 are angled at an angle θ. In one embodiment, an angle of 45° is used, however, any suitable angle allowing the ratchet structures 122 to fully enter the grooves 206 and contact the proximal facing sides 282 of the grooves 206 may be used.

In one alternative embodiment, the proximal member 204 of the anchor top 200 may be free-floating along distal member 202. A stop may then be employed on the proximal end of the distal member 202 to prevent the proximal member 204 from sliding off the anchor top 200. In one embodiment having a free-floating proximal member 204, protrusions on the side of the distal member 202 may be employed to prevent the free-floating proximal member 204 from falling all the way down the distal member 202. The protrusions may be configured to shear off upon application of an axial force to the proximal member 204. Alternatively, other mechanisms may be employed to temporarily prevent the proximal member 204 from sliding all the way down the distal member 202. For example, a weak weld or adhesive may be employed. Alternatively, the proximal member 204 may have a sufficiently tight push fit on the distal member 202 such that it does not move relative to the distal member 202 without application of force.

The proximal member 204 of anchor top 200 is generally cylindrical in shape with a diameter larger than distal member 202. A hole 208 may advantageously be provided in the center of proximal member 204. With reference to FIG. 5B, the bottom of distal member 202 also contains a hole 210. Holes 208 and 210 open into a central bore through the anchor top 200. This central bore allows the wire referred to above to extend through the anchor top 200 to be secured to bore 132 in the anchor bottom 100, thus allowing the anchor bottom 100 to be attached to an anchor inserter while still allowing anchor top 200 to be ratcheted into anchor bottom 100. FIG. 5B also illustrates that proximal member 204 contains a groove 212 in its distal surface. Thus, the distal surface of proximal member 204 is not flat and comprises distally facing surfaces 214 and 216 and side facing surfaces 218 and 220. In some embodiments, some or all of these surfaces may be textured such as with a scallop shape or grooves or other wise altered so as to inhibit movement of suture material pressed against the surfaces. Suitable structures for inhibiting suture movement include, but are not limited to, teeth on the surfaces, bead blasting of the surfaces, etching the surfaces such as through a chemical etch process, use of a rubber o-ring such as around side facing surface 218, and various coatings such as a hydroxyapatite coating, grit coatings, or microencapsulated biocompatible adhesives. In some embodiments, texturing in the distal surfaces of proximal member 204 match texturing in the proximal surfaces of anchor base 100. FIG. 7 is a cross-sectional view of a portion of the proximal member 204 depicting teeth 290 on the side facing surface 218 to inhibit suture movement. The teeth may extend circumferentially around the outside surface of the proximal member 204. It will be appreciated that the embodiments illustrated in FIGS. 5A-5C represent only one possibility; thus, other shapes for the distal surface of proximal member 204 may also be used. FIG. 5C depicts a cross section through the center of anchor top 200. In this figure, the central bore 226 is depicted as are surfaces 214, 216, 218, and 220 and grooves 206.

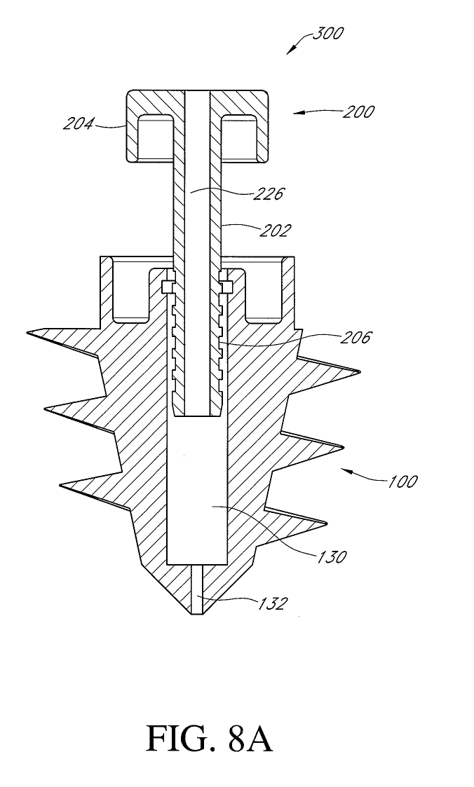

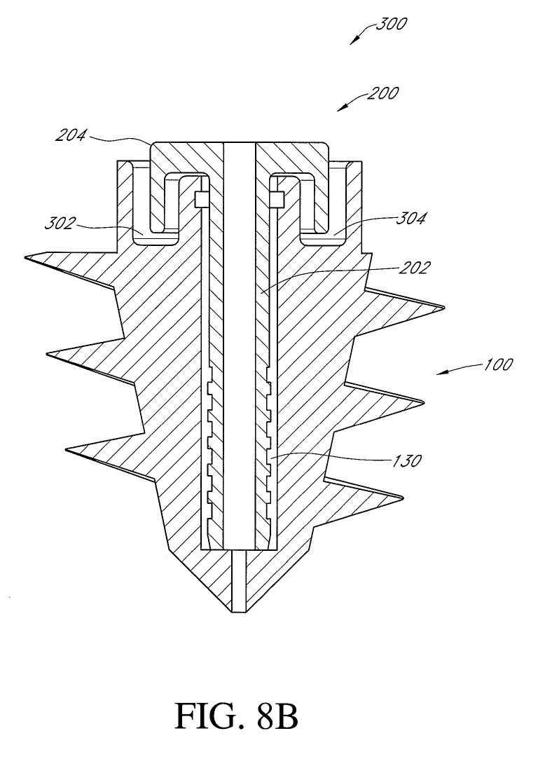

FIGS. 8A and 8B depict cross sections showing how the anchor top 200 may be coupled to anchor base 100 to form the complete anchor 300. In FIG. 8A, the anchor top 200 is coupled to anchor base 100 with the proximal member 204 separated from the anchor base 100. The anchor top 200 is secured to anchor base 100 by distal member 202 extending into central bore 130 of the anchor base 100. The distal member 202 is secured by ratchet structures (not shown) engaging grooves 206 in distal member 202. Central bore 226 in anchor top 200 and central bore 130 in anchor base 100 allow a wire to extend into the top of the anchor 300 and be secured to bore 132. Alternatively, the wire may be secured at other locations within central bore 130. Thus the wire, which can be coupled to an anchor inserter, can hold the entire anchor assembly 300 and still allow anchor top 200 to move relative to anchor base 100 and the wire.

FIG. 8B depicts the anchor assembly 300 with the distal member 202 of anchor top 200 ratcheted all the way into central bore 130 in anchor base 100. In this configuration, it can be seen that proximal surfaces 112, 114, 116, 118, and 120 of the anchor base 100 and distal surfaces 214, 216, 218, and 220 of the proximal member 204 of anchor top 200 form passageways 302 and 304. The size of passageways 302 and 304 are advantageously such that when a suture passes through them, it will be compressed so that it is securely attached to the anchor 300.

In some embodiments, more than one suture may be compressed between the proximal surfaces of the anchor base 100 and the distal surfaces of the proximal member 204. For example, when using the suture geometry depicted in FIG. 3C, two suture lengths may be positioned in the space between the proximal member 204 and anchor base 100 prior to ratcheting the anchor top 200 down.

In one embodiment, an anchor is provided that facilitates the separate attachment of multiple sutures to the same anchor. In one such embodiment, one or more medial members that are similar to the proximal member 204 in FIG. 8A, are positioned on the anchor top 200 to create a “pancake” structure. Separate sutures may be clamped between each medial member, between the upper medial member and the proximal member 204, and between the bottom medial member and the anchor base 100. The anchor may be designed such that the multiple sutures are clamped in place simultaneously. Alternatively, the anchor may be designed such that each suture clamping area may be separately clamped, allowing each suture to be separately tensioned and secured prior to tensioning and securing the other sutures. In some embodiments, multiple sutures may be clamped in each suture clamping area.

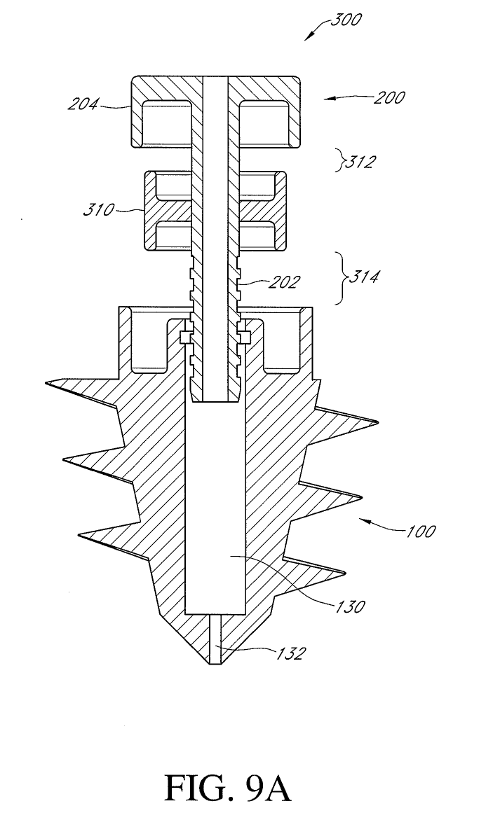

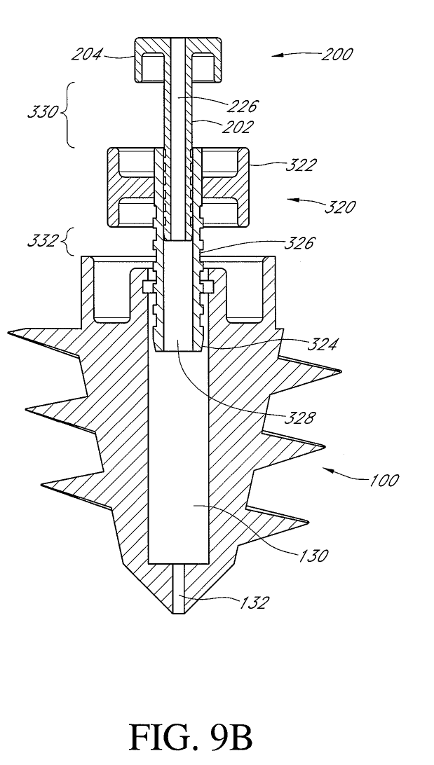

FIG. 9A is a cross-section depicting one embodiment of an anchor allowing the multiple sutures to be clamped simultaneously. Anchor top 200 contains proximal member 204 and medial member 310. Medial member 310 may be free-floating along distal member 202 of the anchor top 200. In one embodiment, medial member 310 may be temporarily fixed relative to the distal member 202 to maintain separation from the proximal member 204 and the anchor base 100. This connection may be through a tight push fit, shearable protrusions, a weak weld, adhesive, or any other suitable mechanism that allows the medial member 310 to move along distal member 202 upon application of force. Sutures may be placed within suture capture zones 312 and 314. Once sutures are placed within the capture zones 312 and 314, force may be applied to the anchor top 200 relative to the anchor base 100 to ratchet the anchor top 200 into the base 100, thereby clamping sutures located in suture capture zones 312 and 314. Those of skill in the art will appreciate that multiple medial members may be utilized to increase the number of capture zones.

FIG. 9B is a cross section depicting one embodiment of an anchor allowing multiple sutures to be clamped in place separately. The anchor in this embodiment contains three parts: an anchor base 100, an anchor middle 320, and an anchor top 200. The anchor middle 320 contains a proximal portion 322 and a distal portion 324. A central bore 328 extends through the anchor middle 320. Similar to the anchor top 200 depicted in FIGS. 5C and 8A, the distal portion 324 of the anchor middle 320 contains grooves 326 for engaging ratchet structures in the central bore 130 of the anchor base. Anchor top 200 is similar to that depicted in FIG. 5C and 8A, with the exception that is sized such that the distal member 202 of the anchor top 200 can fit within and ratchet into the central bore 328 within the anchor middle 320. The combined structure of the anchor creates two suture capture zones 330 and 332. Because both the anchor top 200 and the anchor middle 320 can be ratcheted down, the suture capture zones 330 and 332 can be separately clamped into place. Accordingly, multiple sutures may be secured separately in the capture zones 330 and 332, thereby allowing each suture to be separately tensioned and secured. Those of skill in the art will appreciate that multiple middle anchor elements 320 may be utilized to create even more individually actuatable suture capture zones. Although the proximal portion 204 of the anchor top 200 is depicted in FIG. 9B to be small enough to fit within the upper groove of the anchor middle 320, it will be appreciated that in alternative embodiments, the proximal portion 204 may be sized such as in FIG. 9A so that the anchor middle fits within the groove of the proximal portion 204.

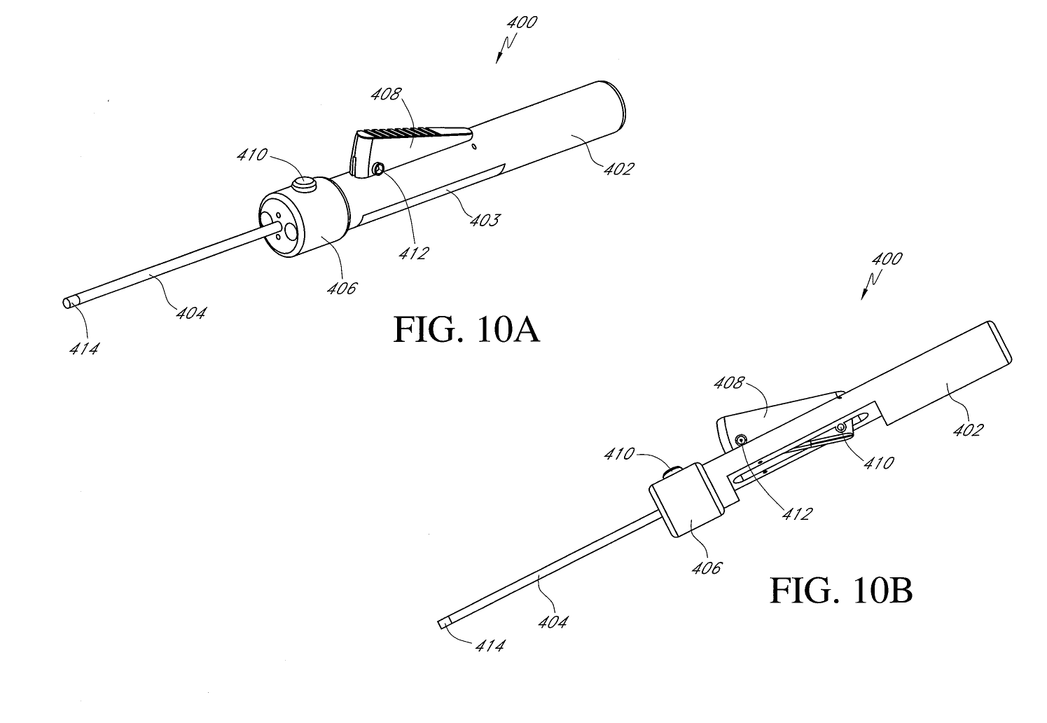

Another embodiment of the present invention is an inserter designed to insert and manipulate an anchor such as described in FIGS. 4A-9B. One such inserter 400 is depicted in FIGS. 10A and 10B. Inserter 400 comprises a handle 402 and an outer tube 404. As depicted in FIG. 10A, the handle 402 comprises a cover 403. Figure 10B depicts the inserter 400 with cover 403 removed. Not depicted in FIGS. 10A and 10B are an inner tube disposed inside outer tube 404 and a wire disposed within the inner tube. As will be described in more detail below, the inner and outer tubes may be used to manipulate an anchor 300 such as that described in FIGS. 4A-9B. The wire may be used to couple the inserter 400 to the anchor 300 as described above. Inserter 400 also comprises an outer tube manipulator 406 and a wire manipulator 408. Outer tube manipulator 406 comprises release button 410. Outer tube manipulator 406 is securely attached to outer tube 404. Outer tube manipulator 406 may move longitudinally relative to handle 402 and the inner tube when release button 410 is pressed. Thus, when outer tube manipulator 406 is moved, outer tube 404 also moves.

Wire manipulator 408 comprises wire grabber 410 to which the wire is attached. The wire extends from wire grabber 410, through handle 402, and then through the inner tube. In one embodiment, wire manipulator 408 also comprises a release button 412. When release button 412 is pressed, the wire manipulator 408 may be pressed into the handle 402 to contact and thus provide additional tension on the wire. When in use, the additional tension causes the anchor base 100 to mover relative to inserter 400. When enough tension is provided to the wire by wire manipulator 408, the wire may break free from the anchor 300 at its attachment point in bore 132 or at some other predetermined location along the wire. It will be appreciated that any suitable breakable attachment means may be used for securing the wire to the anchor 300. For example, the wire may be frictionally secured into bore 132 or it may welded to the anchor base 100 using a weld that is weaker than the wire itself or a portion of the wire where breaking is desired may be weakened. In one embodiment, the wire is notched so as to create a weaker region in the wire that will break upon application of suitable force.

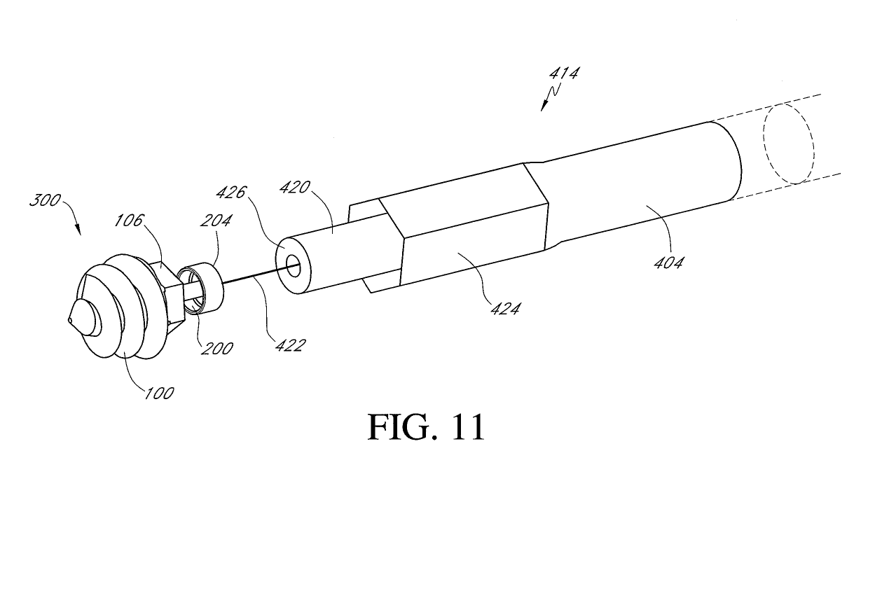

The tip 414 of outer tube 404 is depicted in more detail along with inner tube 420, wire 422, and anchor 300 in FIG. 11. The end of outer tube 404 may comprise a hex nut driver structure 424 for receiving the hex nut structure 106 of anchor base 100. Of course, any other suitable engagement structure can be provided on the inserter 400 and the anchor base 100 in order to facilitate placement of the anchor base 100. Wire 422 extends out of inner tube 420 and into the central bore in the anchor top 200 to attach to anchor base 100 as described above. In some advantageous embodiments, the wire length and tension is adjusted such that the proximal member 204 of anchor top 200 buts against the end 426 of inner tube 420.

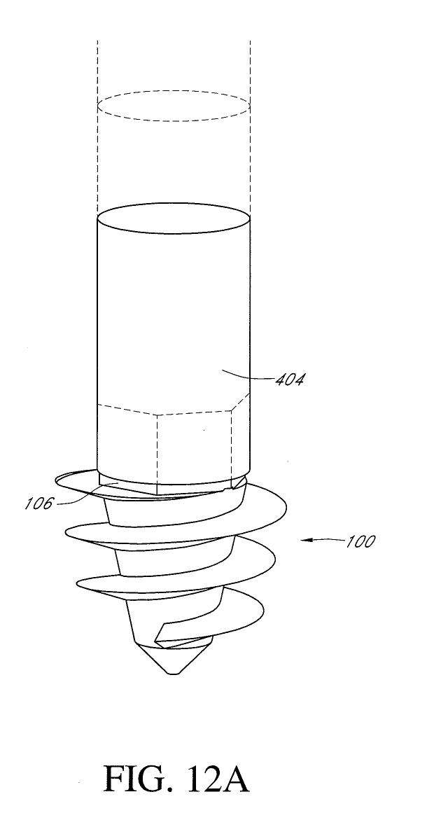

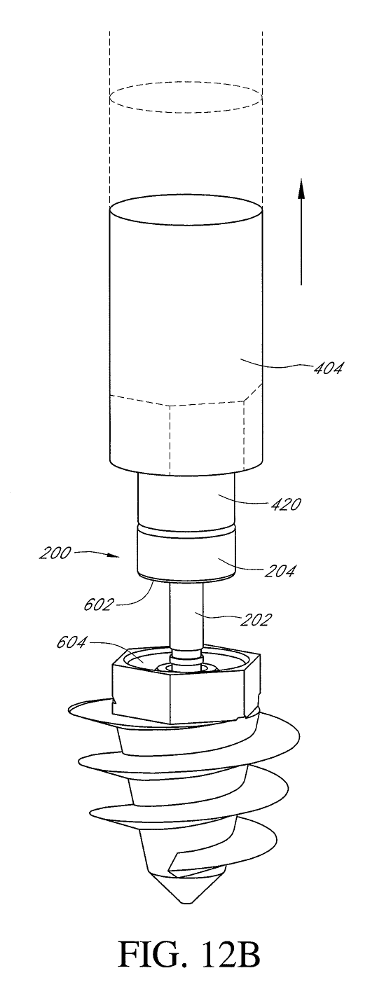

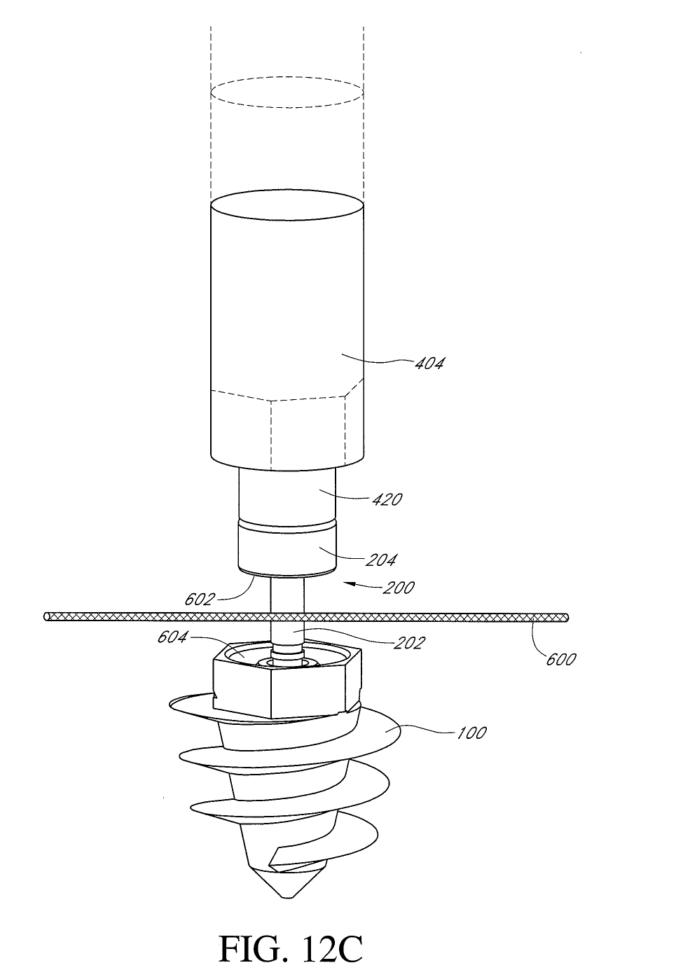

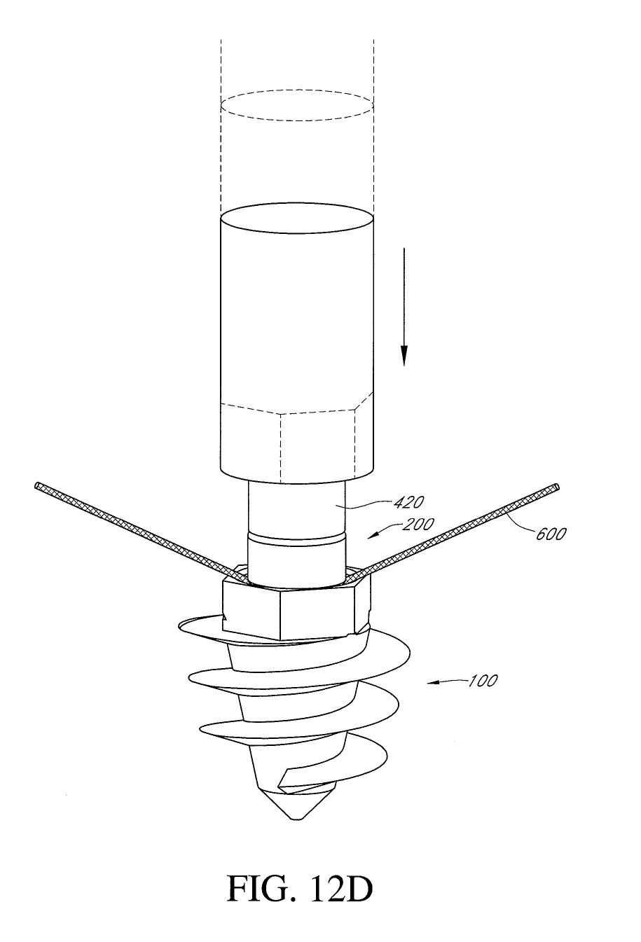

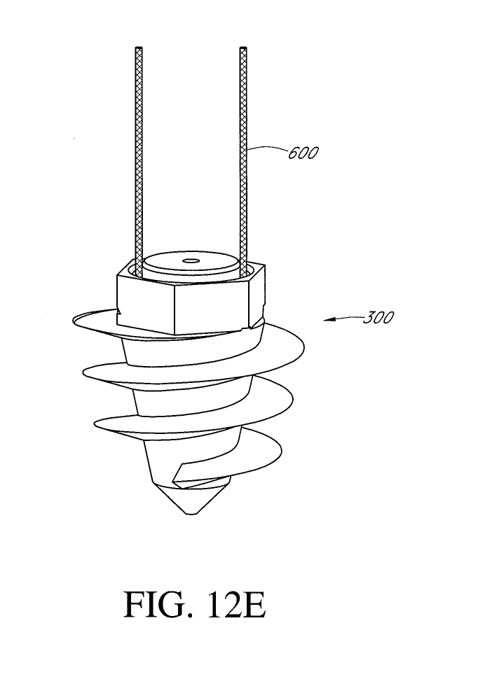

FIGS. 12A through 12E depict how inserter 400 and anchor 300 may be used to insert the anchor 300 into bone and attach a suture to it. FIG. 12A depicts the configuration for inserting the anchor 300 into bone. Outer tube 404 and outer tube manipulator 406 (see FIGS. 10A and 10B) are positioned relative to inner tube 420 and handle 402 (see FIGS. 10A, 10B, and 11) so that the outer tube 404 engages hex nut structure 106 in the anchor base 100. It is advantageous in this configuration for the anchor top 200 to be in a position relative to the anchor base 100 such as depicted in FIG. 8A. In the configuration of FIG. 10A, a surgeon may then screw the anchor base 100 into bone by twisting handle 402 of inserter 400 (see FIGS. 10A and 10B).

After the anchor base 100 is inserted into the bone, the outer tube 404 may be slid backward relative to the inner tube 420 and handle 402 to expose the anchor top 200 such as in FIG. 12B. One or more lengths of suture 600 may then be placed in the space between the distal surface 602 of the proximal member 204 of anchor top 200 and the proximal surface 604 of the anchor base 100 by moving the suture laterally into the space as depicted in FIG. 12C. The suture 600 may be manually tensioned as desired. In some embodiments, tensioning of the suture 600 is aided by pulling the suture 600 against the distal member 202 of the anchor top 200. In embodiments having one or more medial members such as depicted in FIGS. 9A and 9B, sutures may be moved laterally into the multiple suture capture zones.

After appropriate tensioning of suture 600, wire manipulator 408 may be pressed to tension the wire, causing the handle 402 of the inserter 400 and the inner tube 420 to be pulled down towards the anchor base 100 so that inner tube 420 ratchets the anchor top 200 down into the anchor bottom 100 as depicted in FIG. 12D. As the anchor top 200 is pushed axially down, suture 600 will be clamped between the distal surface 602 of the proximal member 204 of anchor top 200 and the proximal surface 604 of the anchor base 100 (see also FIG. 12C). The clamping will force the suture to be compressed within the passageways 302 and 304 depicted in FIG. 8B and thus be secured to anchor 300. The fit between the anchor top 200 and the anchor base 100 in the clamping region is such that the suture 600 is firmly gripped, but is not cut, when it is clamped in place. Appropriate edges that may contact the suture are preferably beveled or rounded to avoid damage to the suture. After anchor top 200 is ratcheted sufficiently into anchor base 100, wire manipulator 408 (see FIGS. 10A and 10B) in inserter 400 may be compressed further to further tension wire 422 (see FIG. 11) such that wire 422 breaks free from its attachment to anchor base 100, thus leaving the anchor 300 free from inserter 400 with suture 600 securely attached as depicted in FIG. 12E.

In embodiments having one or more medial members such as depicted in FIGS. 9A and 9B, when the inner tube 420 is pulled down towards the anchor base 100, it may act to push both the proximal member 200 and medial members 310 or 322 down. Alternatively, tubes in addition to inner tube 420 may be utilized to push multiple separately acutatable anchor elements into place. For example, for the anchor depicted in FIG. 9B, an additional tube may be separately pushed down to contact the medial member 322 and push it down onto the anchor base 100. The inner tube 420 may then be subsequently pressed down to push the anchor top 200 against the proximal end of the medial member 322. Thus, multiple lengths of suture may be separately clamped into place. In some embodiments, multiple wires and wire manipulators may be utilized to effect this result.

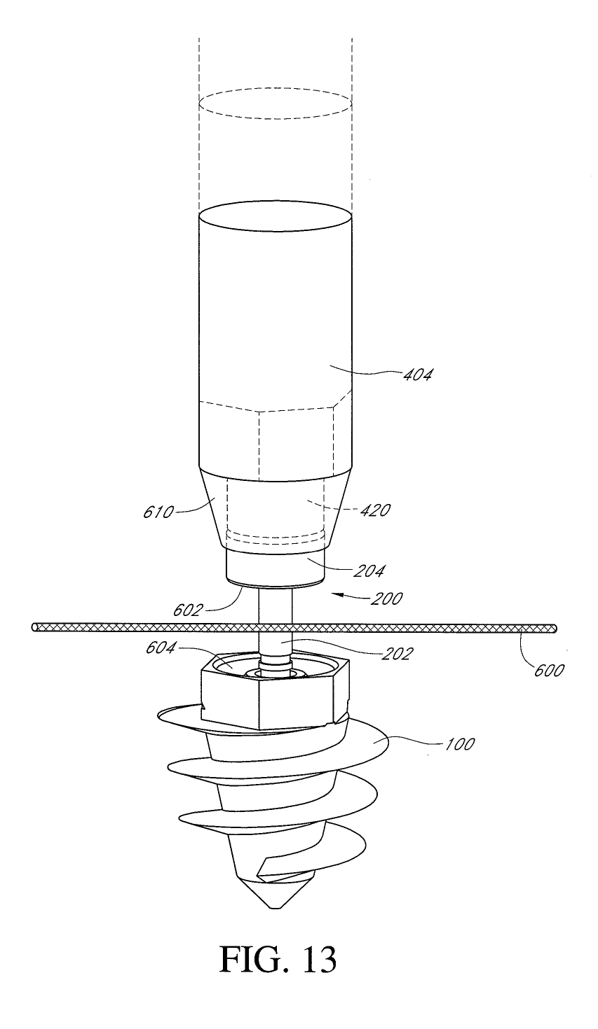

In an alternative embodiment, depicted in FIG. 13, a guide 610 is provided to assist a surgeon in moving the suture 600 laterally into the space between the distal surface 602 of the proximal member 204 of anchor top 200 and the proximal surface 604 of the anchor base 100. Guide 610 covers the abutment region between proximal member 204 of the anchor top 200 and the inner tube 420, thereby preventing a surgeon from accidentally placing the suture 600 between the anchor top 200 and the inner tube 420 instead of the desired capture region. The guide 610 can also inhibit the surgeon from mistakenly placing the suture 600 immediately under the lip of the outer tube 404. In some embodiments, the guide 610 may be tapered as depicted in FIG. 13 to facilitate the suture 600 sliding into place into the capture region.

In one embodiment, the guide 610 is integral with the proximal member 204 of the anchor top 200 such that the proximal member 204 of the anchor top 200 is shaped so that the inner tube 420 engages the proximal member 204 within a recess. In another embodiment, the guide 610 is integral with the inner tube 420 such that the inner tube 420 is shaped to cover the abutment region with a lip. In still another embodiment, the guide 610 is a separate element coupled to the inserter 400. For example, the guide 610 may be a tube positioned between and concentric with the outer tube 404 and the inner tube 420. This middle tube may be stationary relative to the handle 402 of the inserter 400. Thus, when the inner tube 420 ratchets the anchor top 200 down into the anchor bottom 100, the guide/middle tube 610 is displaced axially relative to the inner tube 420 and the anchor top 200. Those of skill in the art will appreciate other guide designs suitable for facilitating accurate placement of the suture 600 laterally into the capture region.

Although a particular inserter device for inserting and manipulating anchor 300 has been described, it should be understood that other inserter designs may be used for manipulating the parts of anchor 300 described above to insert the anchor into bone and secure suture material to the anchor. For example, it may be possible to use separate tools for inserting the anchor and securing the suture material. In addition, in alternative embodiments, the anchor base 100 may be connected to the anchor top 200 throughout the procedure, or the anchor base may be separately inserted into the bone, and the anchor top can be attached thereafter by axially sliding the distal end of the anchor top 200 into the hole 108 in the anchor base 100.

In another alternative embodiment, mechanisms may be employed such that a surgeon does not have to use significant manual force to deploy the clamping mechanism. For example, a shape-memory material such as nitinol (or other shape-memory alloy) may be employed for wire 422. To deploy the anchor top 200 into the anchor base 100, the surgeon may actuate a heating element within the handle 402 (e.g., by pressing a button) to heat the wire 422 and cause it to shorten due to its shape-memory characteristics. Other automatic deployment mechanisms will also be appreciated, such as use of an electric motor.

In one embodiment, the inserter 400 may be powered for the purposes of driving the anchor into the bone. For example, an electric motor may be employed in the handle 402 to rotate the outer tube 404 and hex nut driver structure 424 relative to the handle 402, thereby screwing the anchor base 100 into the bone without the surgeon having to manually rotate the handle 402.

In still other embodiments, visual indicators may be provided in the inserter 400 to provide feedback to a surgeon, such as indicating the deployment state of the anchor or the depth that the anchor is inserted into the bone.

It will be appreciated by those of skill in the art that the anchor 300 and inserter 400 provide a system for easy attachment of a suture to bone. The anchor 300 may be inserted into bone with minimal disruption of surrounding tissue. Only an access route having the diameter of the outer tube 404 and the anchor base 100 is required. Furthermore, the suture can be securely attached to the anchor 300 and tensioned as desired without having to insert additional instrumentation into the site or without performing any cumbersome attachment maneuvers such as knot tying. It should also be appreciated that the general principle illustrated by this system of inserting an anchor into bone without having suture material pre-attached and then attaching suture to the anchor without tying any knots may be implemented using any appropriate system other than the specific embodiments depicted in FIGS. 4A-13.

Tissue and Bone Piercing Anchor

One embodiment is a bone anchor adapted for piercing through the soft tissue and into underlying bone. In one embodiment, the suture material may be pre-attached to the piercing bone anchor so that after implantation, a suture passes from the bone anchor through to the top of the soft tissue for easy passing over the soft tissue. In one embodiment, the piercing bone anchor has two configurations, a first configuration having a small diameter for easy piercing through soft tissue and bone and a second deployed configuration where structures such as protrusions are deployed to prevent the bone anchor from being easily removed from the bone.

In one embodiment, the anchor includes a substantially hollow cylinder having a portion of its walls cut in such a manner so as to allow the cylinder to deform under axial stress and form lateral protrusions. The lateral protrusions may thus prevent the anchor from being easily removed from the bone after deployment. In one embodiment, the anchor comprises a pointed tip coupled to the hollow cylinder for piercing the soft tissue and bone. In one embodiment, suture is pre-attached to the pointed tip inside of the hollow cylinder. In other embodiments, suture is pre-attached at other locations on the piercing anchor, such as at the proximal end of the hollow cylinder.

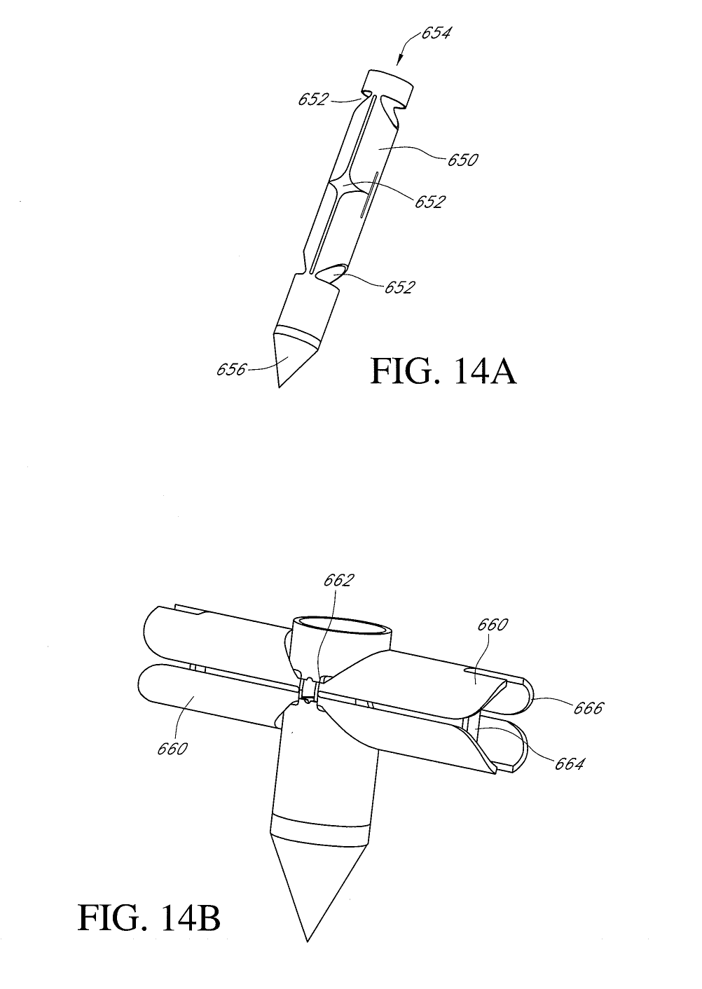

One embodiment of a deployable piercing anchor is depicted in FIGS. 14A and 14B. In FIG. 14A, the anchor is depicted in a pre-deployed state. The anchor includes a substantially hollow cylinder 650 with a plurality of cuts 652 in the side of the cylinder 650. The cylinder 650 is open on one end 654. On the other end, a pointed tip 656 is disposed, allowing the anchor to pierce through soft tissue and bone. In FIG. 14B, the anchor is depicted in a deployed state. Stress is applied in an axial direction such that the cylinder 650 collapses along cuts 652 so as to form two lateral wings 660. The lateral wings 660 prevent the anchor from being removed from the bone. Hinges 662 connect one end of each wing to either the top or the bottom parts of anchor body. These hinges deform and fold, in the plane tangent to the anchor body at that point when the anchor is deployed. A strip of material 664 connects the top and bottom wing on each side of the anchor body, and serves as a hinge between the two as well as aiding in alignment of the wings during deformation. The tips of the wings adjacent to the connecting strip 664 utilize rolling edges 666, which ensure uniform alignment and smooth transition during deformation. Those of skill in the art will appreciate that any number of geometries of cuts in the cylinder 650 may be utilized to create a deformable structure that will produce lateral protrusions upon exposure to stress.

In some embodiments, the lateral wings 660 have a “T” shape as depicted in FIG. 14B. In other embodiments, the lateral wings 660 may be angled relative to the axis of the anchor, such as to form a “Y” shape. Such angling may help prevent anchor pull-out from the bone.

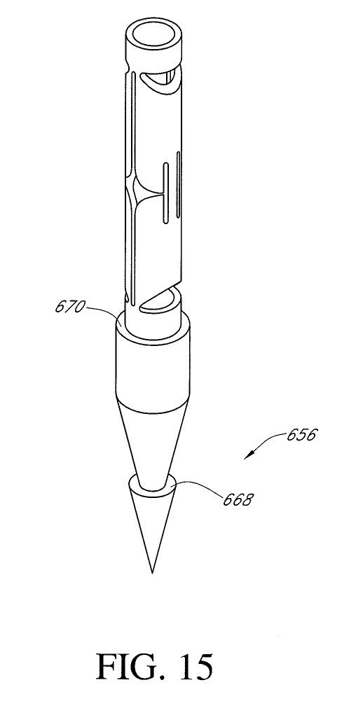

In some embodiments, the tip 656 may include a shoulder, barbs, or other suitable structures for preventing the anchor from sliding out of the soft tissue after the tissue is pierced. Such structures enable the soft tissue to be pierced and then moved using the anchor prior to inserting the anchor into bone. For example, in repairing a torn rotator cuff, it may be desirable to use the anchor to reposition the tendon to the desired location prior to inserting the anchor into bone. FIG. 15 depicts an alternative tip design comprising a shoulder 668 to prevent tip pullout from soft tissue and allow easy manipulation of the soft tissue using the tip. Those of skill in the art will appreciate other structures that may be used to facilitate engagement with soft tissue so that it can be manipulated. Alternatively, the soft tissue may be repositioned using a tissue grasper.

In some embodiments, the piercing anchor can be coupled to an anchor inserter that can be used for inserting the anchor and then deploying the lateral wings. In some embodiments, the anchor inserter includes an inner member attached to the anchor tip and an outer member abutting the top of the deformable cylinder. Insertion force may be applied by the inner member to the anchor tip to insert the anchor. Relative motion between the inner and outer members compresses deformable cylinder causing it to deploy. In one embodiment, the inserter comprises an additional outer sleeve that extends over the cylinder during insertion of the anchor. This outer sleeve acts to prevent the cylinder from deploying prematurely during insertion of the anchor. The outer sleeve may then be retracted prior to deploying the anchor by deformation of the cylinder. In some such embodiments, insertion force may be supplied by the outer sleeve in addition to or alternatively to supplying insertion force with the inner member. The outer sleeve in such embodiments may contact a shoulder on the anchor tip, such as the shoulder 670 depicted in FIG. 15. After anchor deployment, the inserter may be decoupled from the anchor and removed from the body, leaving the preattached suture extending from the anchor.

In an alternative embodiment, mechanisms may be employed such that a surgeon does not have to use significant manual force to deploy the anchor. For example, a shape-memory material such as nitinol (or other shape-memory alloy) or an electric motor may be employed to effect deployment.

In some embodiments, visual indicators may be provided in the inserter handle 700 to provide feedback to a surgeon, such as indicating the deployment state of the anchor or the depth of anchor insertion.

Those of skill in the art will appreciate other mechanisms that could be used for deploying a deployable anchor and providing safety mechanisms to prevent premature deployment.

Example Using a Piercing Anchor and a Suture Capturing Anchor

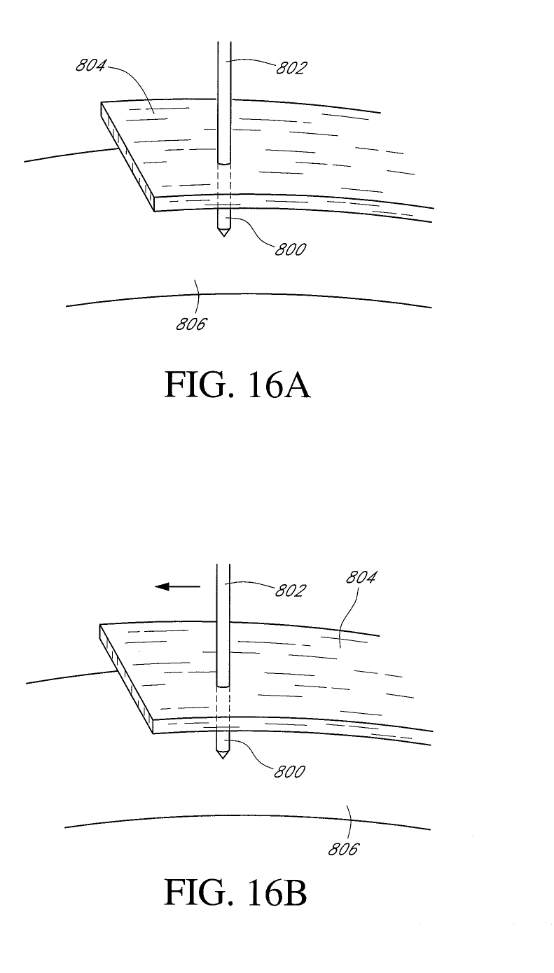

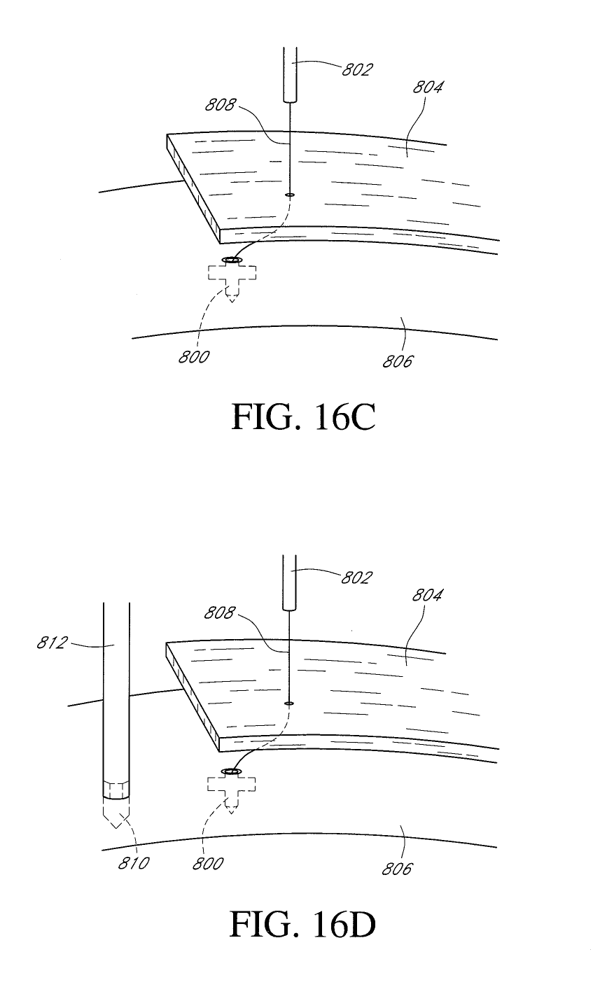

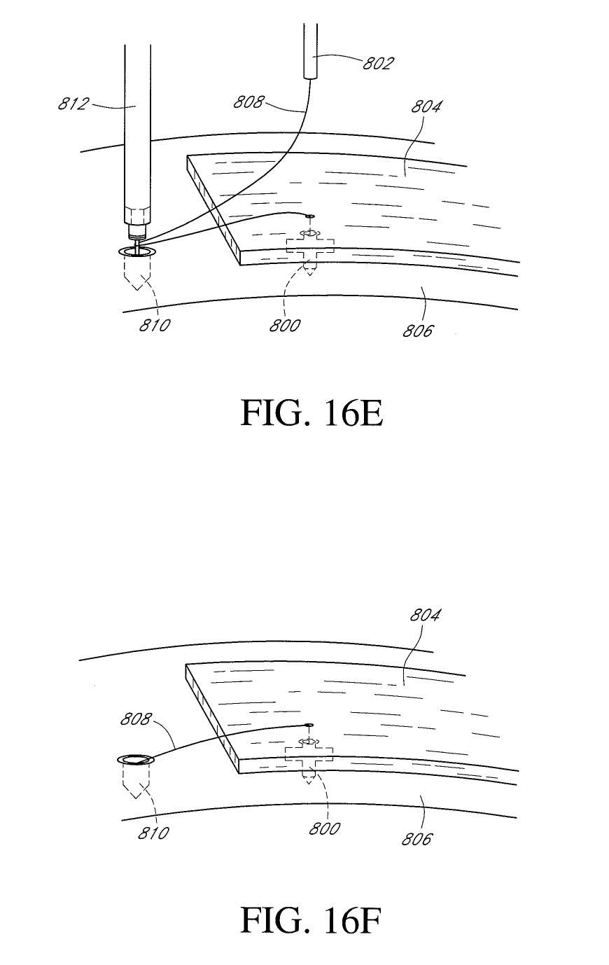

The above-described anchors may be used in a surgical procedure for attaching soft tissue to bone. One example of such a procedure is depicted in FIGS. 16A through 16F. In FIG. 16A, the piercing anchor 800 attached to an anchor inserter 802 as described above is pierced through soft tissue 804 that has become detached from underlying bone 806. In FIG. 16B, the anchor inserter 802 is moved laterally relative to the bone 806 so as to stretch the soft tissue 804 laterally relative to the bone 806. As discussed above, structures such as barbs or shoulders may be employed on piercing anchor 800 to facilitate manipulation of the soft tissue 804. Once the soft tissue 804 has been stretched to the desired position, the anchor 800 is inserted into the bone 806 and the anchor 800 is deployed as described above and the inserter 802 is detached from the anchor 800, leaving a suture 808 attached to the anchor 800 and extending through the soft tissue 804. The anchor 800 may be inserted into bone 806 by tapping on the inserter 802 with a hammer or by any other suitable means of applying axial force. FIG. 16C depicts the deployed anchor 800 with attached suture 808. The suture 808 will extend into the inserter 802.

In some embodiments, systems and methods are used to target the desired insertion site for the piercing anchor 800 prior to insertion. For example, in some embodiments, a suitable needle or probe, such as a spinal needle, may be used to find a desired insertion site. The needle may then be held in place until the inserter 802 with attached piercing anchor 800 is positioned at the needle site. In another embodiment, a peel away sheath is deployed to the desired insertion site using a needle. The peel away sheath then marks the exact site of desired insertion. The piercing anchor 800 may be driven through the center of the peel away sheath. In some embodiments, the sheath may be of smaller diameter than the piercing anchor 800. As the anchor 800 is driven through the sheath, the sheath will split apart. In still another embodiment, a targeting grasper is employed that can grasp the soft tissue and then move it to a desired location. The targeting grasper can be used to hold the soft tissue at the desired location until the piercing anchor 800 is inserted. The targeting grasper can employ a targeting aid for positioning of the anchor 800. For example, the grasper may have an aperture through which the anchor 800 may be inserted. Those of skill in the art will appreciate other methods for targeting the piercing anchor 800 to the desired location.

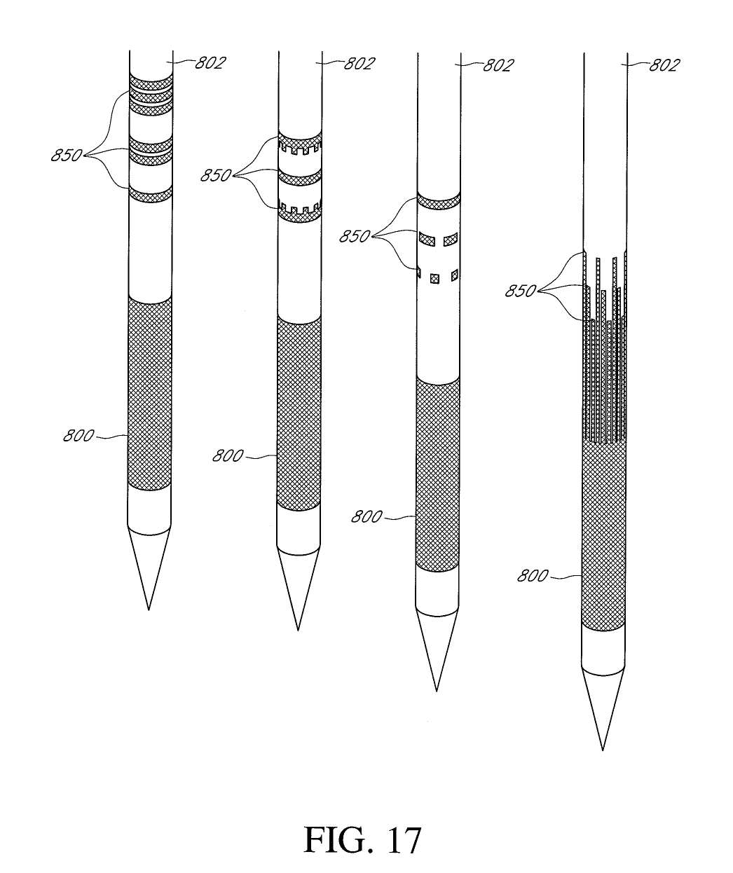

In some embodiments, the inserter 802 may have one or more markings (e.g., on the outer sheath) to aid a surgeon in inserting the anchor 800 to the proper depth. For example, the inserter 802 may have markings that indicate the depth of the anchor relative to the outer surface of the skin (i.e., the surgeon would look at the inserter markings outside of the patient). Other markings on the inserter 802 could be used to indicate the depth of the anchor 800 relative to the top surface of the soft tissue. A surgeon can use these markings to make sure that the anchor 800 is inserted to the proper depth to insure proper deployment as well as prevent over-insertion. FIG. 17 depicts various embodiments of markings 850 on the inserter 802 to show three graduations of depth of the anchor 800. Those of skill in the art will appreciate many other possible marking designs or mechanisms that may be utilized to determine anchor depth.

After insertion of the piercing anchor 800, a suture capturing anchor 810 is inserted into the bone 806 using the inserter 812 as described above, as depicted in FIG. 16D. In FIG. 16E, the inserter 812 is then retracted to expose the suture capturing mechanism. The suture 808 is then passed over the soft tissue 804 and laterally moved into the suture capturing mechanism and tensioned. Finally, as depicted in FIG. 16F, the suture capturing mechanism is deployed to capture the suture 808, the anchor inserter 812 is detached from the anchor 810, and the suture 808 is cut to detach it from the suture inserter 802. The result is a length of suture 808 between the bone anchors 808 and 810 that presses the soft tissue 804 against the bone 806. Multiple anchors and sutures may be used to produce geometries such as depicted in FIGS. 2 and 3 and variations thereof.

It will be appreciated that there are numerous stitches, suture threading patterns, and anchor patterns that may be used to secure soft tissue to bone by the methods and devices described herein. These variations as well as variations in the design of the above described anchor devices and inserter devices are within the scope of the present disclosure.

Methods of Attaching Soft Tissue to Bone

Various embodiments include methods for attaching soft tissue to bone. In some embodiments, the methods include using the bone anchors described above. In one embodiment, a bone anchor is inserted into the bone and then a length of suture is passed over the soft tissue and secured to the anchor after inserting the anchor without tying any knots or without passing the suture through an aperture in the anchor. In some embodiments, the suture is secured to the anchor by laterally moving it into a securing mechanism. In one embodiment, securing the suture to the anchor includes clamping the suture between at least two surfaces on the anchor. In one embodiment, the anchor is not inserted further into the bone after securing the suture to it.

In another embodiment, a first anchor with a suture pre-attached is inserted through the soft tissue and into the bone. The suture may then be passed over the soft tissue and fixedly secured to a second bone anchor. In one embodiment, the first anchor is inserted by directly piercing the soft tissue and the bone. In one embodiment, lateral protrusion may be deployed on the first anchor to prevent the first anchor from being removed. In one embodiment, the suture may be coupled to the second bone anchor prior to insertion and then fixedly secured after insertion. In this context, “coupled” means that the suture is attached to the bone anchor but not fixedly secured, such that the suture can move to some extent relative to the bone anchor. In an alternative embodiment, the suture is not coupled to the second bone anchor during its insertion.

In another embodiment, a first portion of suture is inserted into the proximal surface of the soft tissue. A second portion of the suture (e.g., the portion proximal to the inserted portion) is then passed over the proximal surface of the soft tissue and fixedly secured to a bone anchor. In one embodiment, the procedure may be performed without passing the first portion of the suture back out of the proximal surface of the soft tissue. In one embodiment, this result is accomplished by the first portion of the suture being attached to an anchor that is inserted through the soft tissue and into bone.

One embodiment includes inserting a first anchor with a pre-coupled suture through soft tissue and into bone. The suture may then be passed over the soft tissue and fixedly secured to a second anchor. In one embodiment, the pre-coupled suture is fixedly secured to the first anchor prior to insertion. In an alternative embodiment, the pre-coupled suture can move relative to the first anchor prior to insertion and is fixedly secured after insertion.

In another embodiment, multiple lengths of suture are attached to multiple anchors. In one embodiment at least three anchors are inserted into bone. A first length of suture may be secured between a first and second anchor and a second length of suture may be secured between the first and a third anchor. In one embodiment, the first anchor is positioned beneath the soft tissue and the second and third anchors are positioned laterally to the soft tissue. In an alternative embodiment, the first anchor is positioned laterally to the soft tissue and the second and third anchors are positioned beneath the soft tissue. In some embodiments, the lengths of suture are fixedly secured to the anchor(s) positioned beneath the soft tissue prior to insertion of those anchor(s). In one embodiment, the different lengths of suture may be tensioned separately.

In various embodiments, prior to fixedly securing suture to a bone anchor, it can be tensioned. In one embodiment, tensioning is accomplished by manually pulling on the suture such as by a surgeon grasping the suture using an appropriate instrument and then pulling. In one embodiment, the suture may be pressed against the bone anchor to provide leverage for pulling. For example, the suture may be wrapped partly around a proximal portion of the anchor prior to pulling.

As discussed in more detail above, some embodiments include securing a length of suture between two anchors inserted into the bone. It will be appreciated that these embodiments have the advantage of providing a net compressive force along the length of the suture between the soft tissue and the underlying bone surface, thereby facilitating soft tissue reattachment to the bone. Many alternative anchor-suture systems to those described above may be used to achieve this result.

For example, in one alternative embodiment, suture material is captured by an anchor inserted into the soft tissue rather than captured by an anchor inserted into the bone but not through the soft tissue. In one embodiment, an anchor is inserted into the bone adjacent to a desired reattachment site. Suture is either pre-attached to the anchor or attached after insertion of the anchor. Next, the suture can be passed over the soft tissue and captured by a second anchor that is inserted through the soft tissue and into underlying bone.

Although the invention has been described with reference to embodiments and examples, it should be understood that numerous and various modifications can be made without departing from the spirit of the invention. Accordingly, the invention is limited only by the following claims.

What is claimed is:

1. A suture anchor, comprising:
a distal portion configured to engage bone; and
a proximal portion comprising at least two separate suture capture mechanisms, each configured to secure a separate suture without tying any knots.

2. The anchor of claim 1, wherein each suture capture mechanism comprises first and second surfaces configured to clamp suture therebetween.

3. The anchor of claim 1, wherein the two suture capture mechanisms are configured to secure sutures at different times.

4. The anchor of claim 1, wherein the proximal portion comprises a first, second, third, and fourth surface, wherein the proximal portion is configured to secure at least one suture by clamping between the first and second surfaces and is configured to secure at least one other suture by clamping between the third and fourth surfaces.

5. The anchor of claim 1, wherein each suture capture mechanism defines a suture capture zone configured such that a suture may be moved laterally into the suture capture zone.

6. A suture anchor, comprising:
a distal portion configured to engage bone, the distal portion comprising a proximally facing surface;
a proximal portion comprising a distally facing surface; and
a medial portion comprising a proximally facing surface and a distally facing surface, wherein the anchor is configured to secure at least one suture by clamping between the proximally facing surface of the distal portion and the distally facing surface of the medial portion and adapted to secure at least one other suture by clamping between the distally facing surface of the proximal portion and the proximally facing surface of the medial portion.

7. The anchor of claim 6 configured such that a suture can be clamped between the distally facing surface of the medial portion and the proximally facing surface of the distal portion prior to a suture being clamped between the proximally facing surface of the medial portion and the distally facing surface of the proximal portion.

8. The anchor of claim 6, wherein the proximal portion comprises a shaft positioned within a bore in the distal portion.

9. The anchor of claim 8, wherein the shaft is movable within the bore.

10. The anchor of claim 8, wherein the shaft extends through a bore in the medial portion.

11. The anchor of claim 10, wherein the medial portion is movable relative to the shaft.

12. The anchor of claim 6, wherein the medial portion comprises a shaft positioned within a bore in the distal portion.

13. The anchor of claim 12, wherein the proximal portion comprises a shaft positioned within a bore in the medial portion.

14. A method of attaching two or more sutures to a suture anchor, the method comprising:
moving a first suture laterally into a first suture capture mechanism on the suture anchor;
moving a second suture laterally into a second suture capture mechanism on the suture anchor;
actuating the first suture capture mechanism to secure the first suture to the suture anchor; and
actuating the second suture capture mechanism to secure the second suture to the suture anchor.

15. The method of claim 14, wherein actuating the first suture capture mechanism comprises clamping the first suture between first and second surfaces of the first suture capture mechanism.

16. The method of claim 14, wherein actuating the second suture capture mechanism comprises clamping the second suture between first and second surfaces of the second suture capture mechanism.

17. The method of claim 14, wherein the first and second suture capture mechanisms are actuated simultaneously.

18. The method of claim 14, wherein the first suture capture mechanism is actuated before actuating the second suture capture mechanism.

19. The method of claim 18, wherein the second suture is moved laterally into the second suture capture mechanism after the first suture capture mechanism is actuated.

20. A method of attaching soft tissue to bone, comprising:
inserting a first, second, and third anchor into the bone;
securing a first suture between the first and second anchors over the soft tissue, wherein securing the first suture to the first anchor comprises laterally moving the first suture into a first suture capture mechanism; and
securing a second suture between the first and third anchors over the soft tissue, wherein securing the second suture to the first anchor comprises laterally moving the second suture into a second suture capture mechanism.

21. The method of claim 20, wherein the first suture is secured to the first anchor prior to securing the second suture to the first anchor.

22. The method of claim 20, wherein the first and second sutures are simultaneously secured to the first anchor.

23. The method of claim 20, wherein securing the first suture to the first anchor comprises clamping the first suture between a first and second surface of the first suture capture mechanism.

24. The method of claim 20, wherein securing the second suture to the first anchor comprises clamping the second suture between a first and second surface of the second suture capture mechanism.

25. A suture anchor, comprising:
an anchor base configured to engage bone, wherein the anchor base comprises a bore having an opening on a proximal end of the anchor base, wherein the inner surface of the bore comprises angled protrusions extending in a direction away from the opening; and
an anchor top comprising a shaft configured to fit within the bore, wherein the shaft comprises circumferential grooves in its outer surface, wherein the circumferential grooves have a profile shaped such that when the angled protrusions extend into the grooves, the protrusions are not significantly deflected from their orientation prior to insertion of the shaft into the bore.

26. The anchor of claim 25, wherein the circumferential grooves comprise surfaces angled relative to the outer surface of the shaft at approximately the same angle as the protrusions such that when the protrusions extend into the grooves, the protrusions are aligned with angled surfaces.

27. The anchor of claim 26, wherein the angle of the angled surfaces is approximately 45°.

28. The anchor of claim 25, wherein the anchor top comprises a member positioned on the proximal end of the shaft having a distally facing surface such that a suture may be clamped between the distally facing surface and the anchor base when the shaft is inserted into the bore.

29. A suture anchor, comprising:
an anchor base configured to engage bone, wherein a proximal end of the anchor base comprises a circular groove; and
an anchor top comprising a circular lip configured to fit inside the circular groove such that a suture can be clamped between the surfaces of the lip and the surfaces of the groove, wherein a least one surface of the lip or the groove comprise teeth configured to engage the suture.

30. A suture anchor inserter, comprising:
a handle;
a tube coupled to the handle configured to engage an actuatable suture anchor;
a shape memory alloy wire coupled to the handle and coupled to the actuatable suture anchor; and
a heating element configured to heat the shape memory alloy wire, thereby causing the wire to contract relative to the tube and actuate the suture anchor.

31. The inserter of claim 30, wherein the shape memory alloy wire is a nitinol wire.

32. The inserter of claim 30, wherein the actuatable suture anchor comprises an anchor base and an anchor top configured to actuate by pressing the anchor top down onto the anchor base.

33. The inserter of claim 30, wherein the actuatable suture anchor comprises a deformable anchor configured to deform under axial force to form lateral wings.

34. A suture anchor inserter, comprising:
a handle;
a tube coupled to the handle configured to engage an actuatable suture anchor;
a wire coupled to the handle and coupled to the actuatable suture anchor; and
a motor configured to move the wire axially relative to the tube, thereby actuating the suture anchor.

35. The inserter of claim 34, wherein the actuatable suture anchor comprises an anchor base and an anchor top configured to actuate by pressing the anchor top down onto the anchor base.

36. The inserter of claim 34, wherein the actuatable suture anchor comprises a deformable anchor configured to deform under axial force to form lateral wings.
